(19) United States
(12) Patent Application Publication
(10) Pub. No.: US 2018/0055091 A1
(43) Pub. Date: Mar. 1, 2018

(54) Vaporization Device for Use in Vaporization of Phyto Material

(71) Applicant: MICHAEL TRZECIESKI, TORONTO (CA)

(72) Inventor: MICHAEL TRZECIESKI, TORONTO (CA)

(21) Appl. No.: 15/683,395

(22) Filed: Aug. 22, 2017

Related U.S. Application Data
(60) Provisional application No. 62/377,841, filed on Aug. 22, 2016.

(51) Int. Cl. A24F 47/00; A61M 11/04; A61M 15/00; A61M 15/06

(52) U.S. Cl. CPC A24F 47/008; A61M 11/042; A61M 15/0023; A61M 2205/3673; A61M 15/06; A61M 2205/8206; A61M 15/009

(57) ABSTRACT

A novel vaporization device is disclosed for vaporizing of phyto material, wherein it has an atomizer for operating in a first mode of operation, which is a closed mode and a second mode of operation which is an opened mode. The atomizer is coupled with a rotating member having a rotational axis and rotationally coupled with the housing for rotating about the rotational axis between the first and second modes of operation.

### CROSS REFERENCE TO RELATED APPLICATION

This application benefits from the priority of U.S. Provisional Applications 62/377,841 filed on Aug. 22, 2016, which is incorporated herein by reference and also is a Continuation In Part of U.S. Patent Application 62/263,751 filed on Dec. 7, 2016.

### TECHNICAL FIELD OF THE INVENTION

The technical field relates to a device for vaporization of phyto materials and more specifically to a device for aromatherapy vaporization.

### BACKGROUND OF THE INVENTION

Aromatherapy generally uses essential oils, which are extracted from phyto materials, such as leaves of plants, for therapeutic benefits. These essential oils are either massaged into the skin or can be inhaled. In some cases the phyto materials are heated in order to released the essential oils therefrom. By heating these phyto materials at predetermined temperatures, essential oils and extracts are boiled off, depending upon the temperature at which these phyto materials are heated, an aroma or vapor is given off, which is then inhaled by a user for its therapeutic benefits. Devices that provide such operation are generally known as vaporizers. Different phyto materials release vapors at different temperatures. Some release vapors at 120 degrees Celsius, whereas others at 220 degrees Celsius. Ideally the predetermined temperature is less than a combustion temperature of the phyto material or loose leaf material.

More recently, there has been a trend in extracting of essential oils from the ground leaf materials and using these extracts inside vaporization devices. These extracts are also usually in a consistency honey or wax and are very messy to work with. They typically leave behind a residue, are sticky to work with and generally lack in cleanliness when they are used. Furthermore there are issues with prior art devices that are not as discreet as one would hope. As well prior art devices may at times be hot when placed back into a pocked of a user after utilization thereof. In addition when they are placed back into a pocked of a user a mouthpiece that thereof that is used for inhalation may become dirty because its not protected or covered. In some cases these prior art devices have short battery lives because of their thin form factors.

It is therefore an object of the invention to provide an aromatherapy vaporization device that overcomes the aforementioned deficiencies.

### SUMMARY

In accordance with the embodiments of the invention there is provided A vaporization device for vaporizing of phyto material comprising: an atomizer comprising a heating chamber for receiving of the phyto material, the atomizer comprising a inhalation aperture disposed at a first end thereof and a coupling mechanism disposed at a second end thereof, opposite the first end, the atomizer for operating in a first mode of operation—a closed mode—and a second mode of operation—a second mode, the atomizer comprising a heating element disposed within the heating chamber, the heating element for providing of heat to the phyto material using at least one of convection and conduction; a housing having a first portion and a second portion, the first portion having a recessed channel that is open on at least one side for receiving of the atomizer substantially disposed therein in the first mode of operation and for having the atomizer other than disposed within the first portion in the second mode of operation; a rotating member having a rotational axis and rotationally coupled with the housing for rotating about the rotational axis between the first and second modes of operation; a pushbutton switch mechanically coupled with the housing and the rotating member, the pushbutton switch for facilitating movement of the rotating member about the rotational axis and for locking of the rotating member in its position in at least one of the first and second modes of operation, wherein the first mode of operation the rotating member is approximately at a zero degrees orientation with respect to the housing and in the second mode of operation the rotating member is at a greater than forty five degrees orientation with respect to the housing; a battery and a control circuit disposed within the housing the control circuit electrically coupled with the battery and the coupling mechanism.

In accordance with the embodiments of the invention there is provided A vaporization device for vaporizing of phyto material comprising: an atomizer comprising a heating chamber for receiving of the phyto material, the atomizer comprising a inhalation aperture disposed at a first end thereof and a coupling mechanism disposed at a second end thereof, opposite the first end, the atomizer for operating in a first mode of operation—a closed mode—and a second mode of operation—a second mode, the atomizer comprising a heating element disposed within the heating chamber, the heating element for providing of heat to the phyto material using at least one of convection and conduction; a housing having a first portion and a second portion, the first portion having a recessed channel that is open on at least one side for receiving of the atomizer substantially disposed therein in the first mode of operation and for having the atomizer other than disposed within the first portion in the second mode of operation; a rotating member having a rotational axis and rotationally coupled with the housing for rotating about the rotational axis between the first and second modes of operation; a pushbutton switch mechanically coupled with the housing and the rotating member, the pushbutton switch for facilitating movement of the rotating member about the rotational axis and for locking of the rotating member in its position in at least one of the first and second modes of operation, wherein the first mode of operation the rotating member is approximately at a zero degrees orientation with respect to the housing and in the second mode of operation the rotating member is at a greater than forty five degrees orientation with respect to the housing; a battery and a control circuit disposed within the housing the control circuit electrically coupled with the battery and the coupling mechanism; at least one electrical switch disposed between the housing and the rotating member, the at least one electrical switch electrically disposed between the control circuit and the coupling mechanism, the electrical switch for being in an electrically opened position when in the first mode of operation where electric current other than flows from the control circuit to the heating element and in an electrically closed position in the second mode of operation where electric current flows from the control circuit to the heating element.

In accordance with the embodiments of the invention there is provided a vaporization device for vaporizing of phyto material comprising: an atomizer comprising a heating chamber for receiving of the phyto material, the atomizer comprising a inhalation aperture disposed at a first end thereof and a coupling mechanism disposed at a second end thereof, opposite the first end, the atomizer for operating in a first mode of operation—a closed mode—and a second mode of operation—a second mode, the atomizer comprising a heating element disposed within the heating chamber, the heating element for providing of heat to the phyto material using at least one of convection and conduction; a housing having a first portion and a second portion, the first portion having a recessed channel that is open on at least one side for receiving of the atomizer substantially disposed therein in the first mode of operation and for having the atomizer other than disposed within the first portion in the second mode of operation; a rotating member having a rotational axis and rotationally coupled with the housing for rotating about the rotational axis between the first and second modes of operation; a pushbutton switch mechanically coupled with the housing and the rotating member, the pushbutton switch for facilitating movement of the rotating member about the rotational axis and for locking of the rotating member in its position in at least one of the first and second modes of operation, wherein the first mode of operation the rotating member is approximately at a zero degrees orientation with respect to the housing and in the second mode of operation the rotating member is at a greater than forty five degrees orientation with respect to the housing; a battery and a control circuit disposed within the housing the control circuit electrically coupled with the battery and the coupling mechanism; a first electrical contact a second electrical contact, wherein the first electrical contact comprises a ground connection and the second electrical contact comprises a positive connection from the control circuit; and, a third electrical contact and wherein the atomizer comprises a temperature sensor disposed proximate the heating element for sensing a temperature of the heating element and for providing of a temperature signal through the third electrical contact to the control circuit for varying a power provided from the battery to the heating element.

### BRIEF DESCRIPTION OF THE DRAWINGS

FIG. 1A illustrates a vaporization device for vaporizing of phyto material in accordance with a first embodiment of the invention and in a first mode of operation;

FIG. 1B illustrates a vaporization device for vaporizing of phyto material in accordance with a first embodiment of the invention and in a second mode of operation;

FIG. 1C illustrates a vaporization device for vaporizing of phyto material in accordance with a first embodiment of the invention and in a second mode of operation with internal details;

FIG. 1D illustrates a vaporization device for vaporizing of phyto material in accordance with a first embodiment of the invention and in a first mode of operation with a pick tool;

FIG. 1E illustrates a vaporization device for vaporizing of phyto material in accordance with a first embodiment of the invention and shows a spring with a rotational member removed for clarity;

FIG. 1F illustrates a coupling mechanism in detail;

FIG. 1G illustrates at least one electrical switch disposed between a housing and a rotating member;

FIG. 1H illustrates first and second air gaps as part of the vaporization device for vaporizing of phyto material in accordance with the first embodiment of the invention and in the first mode of operation ;

FIG. 1I illustrates a coupling mechanism in accordance with another embodiment of the invention with first, second and third electrical contacts formed therein;

FIG. 1J illustrates a schematic of a heating element coupled with the at least one electrical switch 108a and a control circuit;

FIG. 1K illustrates a schematic of a first electrical contact and a second electrical contact and a third electrical contact 9993 coupled with a control circuit and a heating element and a temperature sensor;

FIG. 2A illustrates a vaporization device for vaporizing of phyto material in accordance with a second embodiment of the invention and in a first mode of operation;

FIG. 2B illustrates a vaporization device for vaporizing of phyto material in accordance with a second embodiment of the invention and in a second mode of operation;

FIG. 2C illustrates a vaporization device for vaporizing of phyto material in accordance with a second embodiment of the invention and in a second mode of operation with an atomizer removed from the rotating member.

### DETAILED DESCRIPTION OF EMBODIMENTS OF THE INVENTION

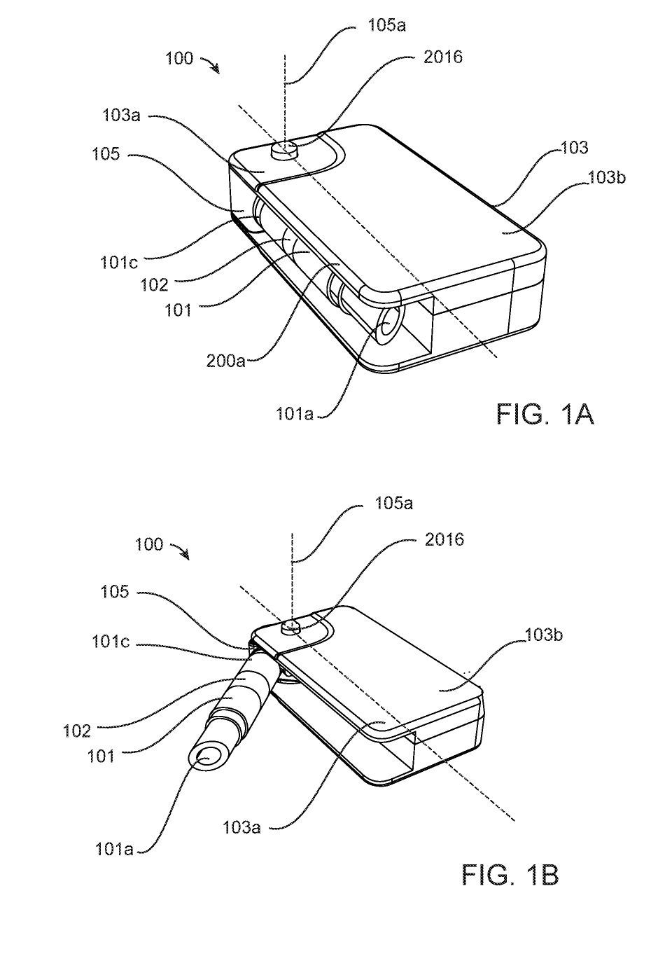

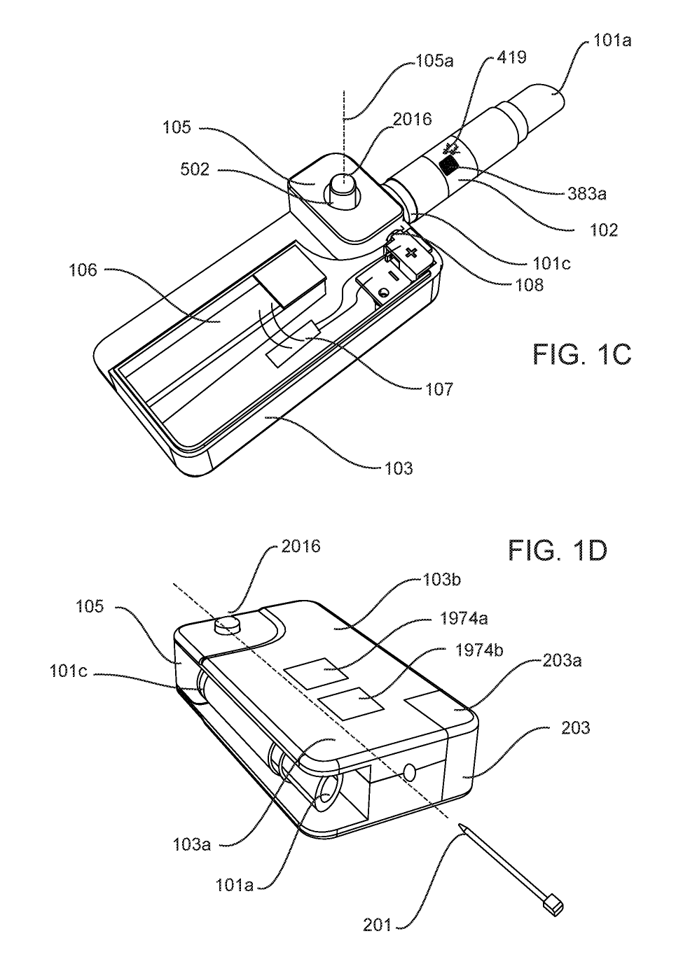

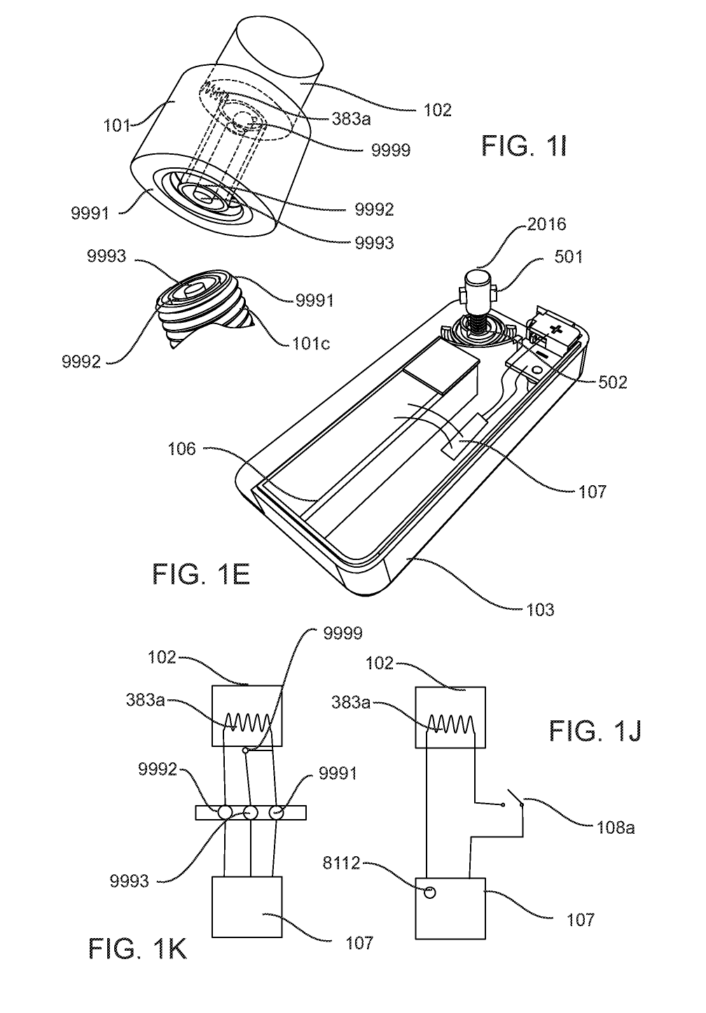

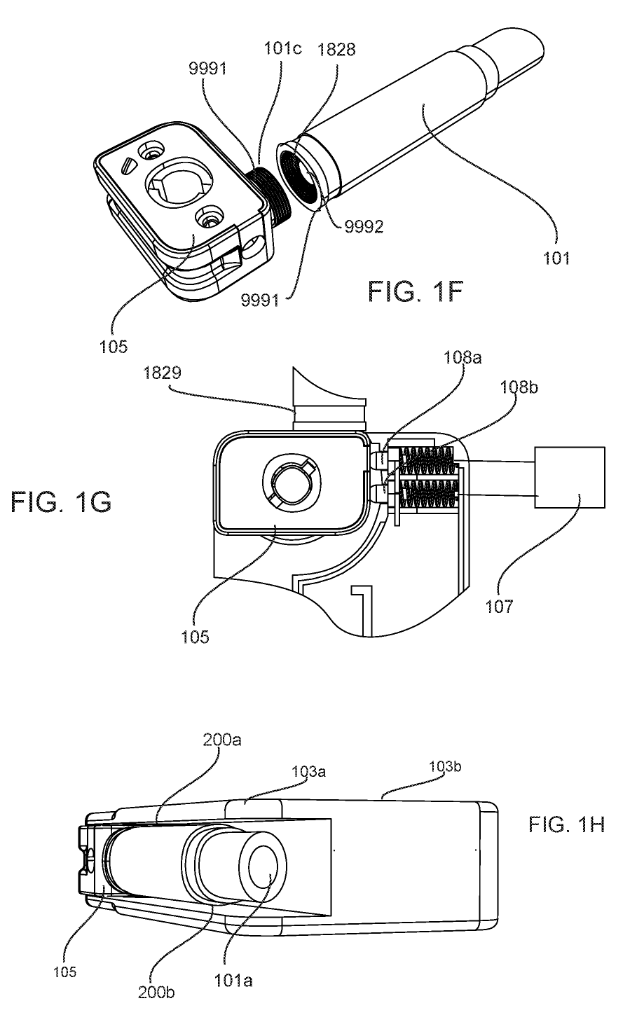

FIG. 1A illustrates a vaporization device for vaporizing of phyto material 100 in accordance with a first embodiment of the invention and in a first mode of operation. The vaporization device for vaporizing of phyto material 100 is formed from an atomizer 101 comprising a heating chamber 102 for receiving of the phyto material 419, the atomizer 101 comprising a inhalation aperture 101a disposed at a first end 101b thereof and a coupling mechanism 101c disposed at a second end 101d thereof, opposite the first end 101a.

The atomizer 101 is for operating in a first mode of operation as is shown in FIG. 1A, which is also known as a closed mode and for operating in a second mode of operation a second mode, which is a second mode or an open mode, as is shown in FIG. 1B.

Referring to FIG. 1C, the atomizer 101 comprises a heating element 383a disposed within the heating chamber 102, the heating element 383a for providing of heat to the phyto material 419 using at least one of convection and conduction. The heating element 383a is envisaged as being a coil heating element wrapped around a wick or a ceramic heating element or a hot air convection heating element as are known in the art.

Referring to FIGS. 1A and 1B, a vaporization device 100 for vaporizing of phyto material 100 is formed from a housing 103 having a first portion 103a and a second portion 103b adjacent the first portion 103a. The first portion 103a having a recessed channel that is open on at least one side (FIG. 1B) for receiving of the atomizer 101 substantially disposed therein in the first mode of operation (FIG. 1A) and for having the atomizer 101 other than disposed within the first portion 103a in the second mode of operation (FIG. 1B).

As shown in FIG. 1C, a rotating member 105 having a rotational axis 105a is provided and rotationally coupled with the housing 103 for rotating about the rotational axis 105a between the first and second modes of operation. In addition FIG. 1C illustrates phyto material 419 disposed within the heating chamber 102 proximate the heating element 383a.

As is evident in FIGS. 1A and 1B, pushbutton switch 2016 is mechanically coupled with the housing 103 and the rotating member 105, the pushbutton switch 2016 for facilitating movement of the rotating member 105 about the rotational axis 105a and for locking of the rotating member 105 in its position in at least one of the first and second modes of operation, wherein the first mode of operation the rotating member 105 is approximately at a zero degrees orientation with respect to the housing 103 (FIG. 1A) and in the second mode of operation the rotating member 105 is at a greater than forty five degrees orientation with respect to the housing 103 (FIG. 1B). As such, in the first mode of operation, the atomizer 101 is protected from at least two sides by the housing first portion 103a.

In addition, FIG. 1A illustrates a first air gap 200a formed between atomizer 101 and the housing first portion 103a in the first mode of operation, wherein the at least a first air gap shields the user in conjunction with the housing first portion 103a from a hot atomizer after use when the vaporization device 100 is placed in a pocket of the user, from at least two sides. The example the recessed channel is an “L” shaped channel or a “U” shaped channel, where an “L” shaped channel provides protection for the atomizer from at least two sides and a “U” shaped channel provides protection for the atomizer from at least three sides. Of course, these “L” and “U” shaped channels may or may not be continuous and may have holes in them or other features to allow for airflow therein.

Referring to FIG. 1C a battery 106 and a control circuit 107 disposed within the housing 103 and preferably within the housing second portion 103a. The control circuit 107 is electrically coupled with the battery 106 and the coupling mechanism 101c, where the coupling mechanism is illustrated in more detail in FIG. 1F.

FIG. 1D illustrates a vaporization device 100 for vaporizing of phyto material 419 in accordance with a first embodiment of the invention and in a first mode of operation with a pick tool 201, the a pick tool 201 for being disposed within the housing second portion 103b, the pick tool 201 for at least one of stirring the phyto material 419 within the heating chamber 102 and for removing of spent phyto material from the heating chamber 102. In addition, a phyto material storage container 203 is disposed as part of the housing second portion 103b for storing of phyto material 419 therein and having a removable lid 203a for accessing of the phyto material 419 stored therein for optionally being loaded into the heating chamber 102. Optionally, control buttons 1974a and 1974bcoupled with the control circuit 107 are provided for increasing and decreasing an electrical power that is applied to the atomizer 101 from the battery 106.

FIG. 1E illustrates a vaporization device 100 for vaporizing of phyto material 419 in accordance with a first embodiment of the invention having a spring 501, the spring 501 for being compressed in the first mode of operation when the atomizer 101 travels from the second mode of operation back to the first mode of operation and the spring 501 pressing against a pushbutton switch 2016 against the rotating member 105 upon the pushbutton switch 2016 being activated by the user it moves away from a locking portion 502 of the rotating member 105 and spring releases stored energy and the rotating member 105 rotates about its rotational axis 105a and the atomizer 101 leaves the housing first portion as the second mode of operation. The rotational member 105 in this case is removed for clarity.

FIG. 1F illustrates the coupling mechanism 101c in more detail, where the coupling mechanism 101c comprises a 510 thread 1828 for threaded coupling of the atomizer 101 to the rotating member 105. This coupling mechanism 101c advantageously works with standard off the shelf 510 threads that have the first and second electrical contacts, as shown in FIG. 1F. Many atomizers that use the 510 threads are known in the art. Some of these atomizers are pre filled with phyto material extract while others allow for a user to insert their own phyto material extract into the heating chamber 102.

Referring to FIG. 1G, the coupling mechanism 101c comprises other than a 510 thread 1828 and comprises a magnetic coupling 1829 for magnetic coupling of the atomizer 101 to the rotating member 105. In some cases this may be more preferable than having a threaded connection for facilitating a quicker release and for facilitating swapping of the atomizers. Furthermore, at least one electrical switch 108a is disposed between the housing 103 and the rotating member 105, the at least one electrical switch electrically disposed between the control circuit 107 and the coupling mechanism 101c, the electrical switch 108a for being in an electrically opened position when in the first mode of operation where electric current other than flows from the control circuit 107 to the heating element 383a and in an electrically closed position in the second mode of operation where electric current flows from the control circuit to the heating element 383a. Having this electrical switch preferably doesn't allow for the atomizer to be accidentally energized when in the first position, an exemplary schematic is shown in FIG. 1J and is discussed in more detail below.

FIG. 1H illustrates first and second air gaps, 200a and 200b, respectively. The first air gap 200a is shown in FIG. 1A and a second air gap 200b is formed between atomizer 101 and the housing first portion 103a in the first mode of operation, wherein the second air gap 200b shields the user in conjunction with the housing first portion 103a from an opposite side of the first air gap 200a from a hot atomizer after use when the vaporization device 100 is placed in a pocket of the user. There is a first air gap present in the case of an “L” shaped channel and first and second air gaps present when there is a “U” shaped channel.

FIG. 1I illustrates a coupling mechanism 101c in accordance with a second embodiment of the invention with first, second and third electrical contacts, 9991, 9992, 9993 are formed therein. Having the three electrical contacts allows for a common ground, an atomizer power and a sensing ability. Within the atomizer 101, a temperature sensor 9999 is disposed proximate the heating element 383a for sensing a temperature of the heating element 383a and for providing of a temperature signal through the third electrical contact 9993 to the control circuit 103 for varying a power provided from the battery 106 to the heating element 383a. If an atomizer with a third electrical contact 9993 as described in the embodiments of the invention is electrically coupled with the coupling mechanism 101c, then the benefits of this third electrical contact 9993 are realized. However a state of the art dual electrical contact atomizer is made to function with this coupling mechanism 101c, however the benefits of the temperature sensing will not be realized. Optionally, where temperature sensing with a temperature sensor 9999 are not possible, a Wheatstone bridge 8612 (FIG. 1J), as is known in the art, is utilized to sense a resistance of the heating element 383a, and the control circuit 107 provides power to the heating element 383a and then selectively allows for the Wheatstone bridge to measure 8612 to measure the resistance of the heating element 383a.

As is shown in FIG. 1J, the heating element 383a is coupled with the at least one electrical switch 108a and the control circuit 107, where another terminal of the heating element 383a is coupled directly to the control circuit 107. Optionally of course two electrical switches are provided, as is shown in FIG. 1G, in the form of a second electrical switch 108b.

FIG. 1K illustrates, the first electrical contact 9991 the second electrical contact 9992 and the third electrical contact 9993 coupled with the control circuit 107, where for example the temperature sensor is disposed between the first and second electrical contacts 9991 and 9992 and the heating element is disposed between the first and third electrical contacts, 9991 and 9993. The heating element 383a is coupled with the at least one electrical switch 108a and the control circuit 107, where another terminal of the heating element 383a is coupled directly to the control circuit 107. Optionally of course two electrical switches are provided, as is shown in FIG. 1G, in the form of a second electrical switch 108b.

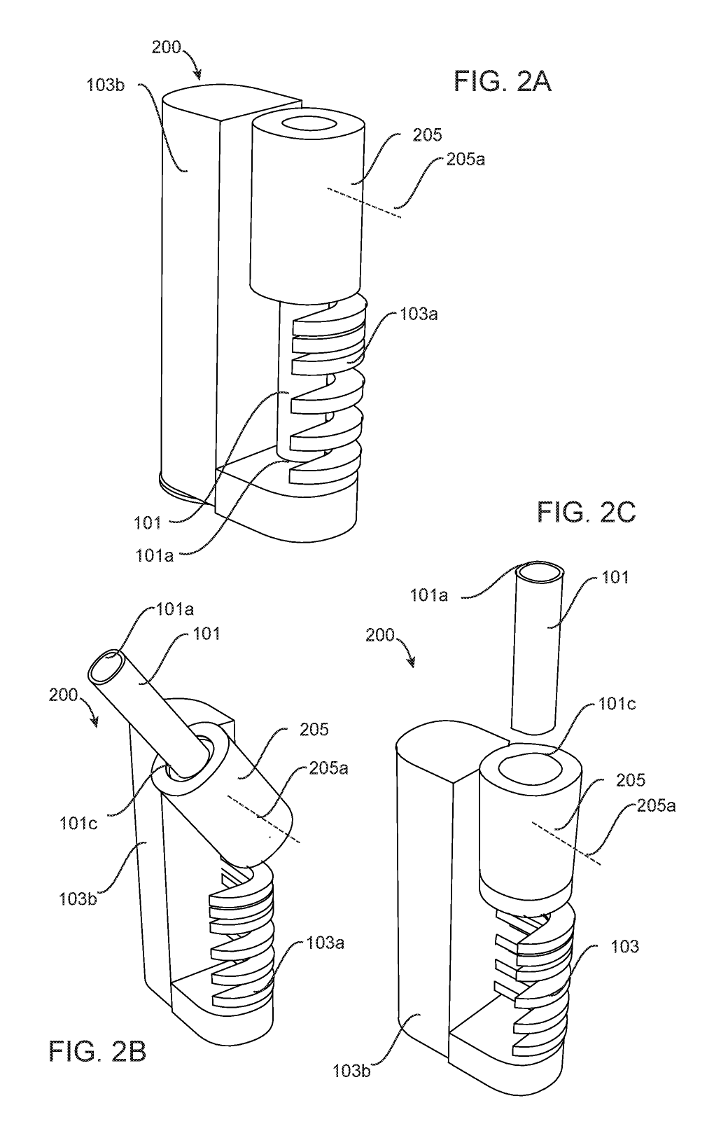

FIG. 2A illustrates a vaporization device 200 for vaporizing of phyto material in accordance with a second embodiment of the invention and in a first mode of operation, the rotating member 205 having a rotational axis 205a and rotationally coupled with the housing 103 for rotating about the rotational axis 205a between the first and second modes of operation. In this embodiment, the rotational axis 205a is at an angle to the rotational axis 105a of the first embodiment.

FIG. 2B illustrates a vaporization device 200 for vaporizing of phyto material 419 in accordance with a second embodiment of the invention and in a second mode of operation.

FIG. 2C illustrates a vaporization device 200 for vaporizing of phyto material 419 in accordance with a second embodiment of the invention and in a second mode of operation with an atomizer removed from the rotating member. In the second embodiment, the first portion 103a having a recessed channel is optionally segmented and has gaps that are in the form or protective ribs that still protect the atomizer in the second mode of operation as well as to provide for improved cooling thereof as there are more spaces for airflow between the ribs. In this case this is an example of a “U” channel.

Advantageously, the vaporization device in accordance with the embodiments of the invention protects the atomizer 101, which may in cases contain glass and ceramic parts, from external shock, such as is the case when vaporization device is possibly dropped. Further advantageously the first embodiment of the invention resembles a flip out car key so that its very discreet and optionally works with many various atomizers already on the market. Various thicknesses for the vaporization device are envisaged to allow for fatter and thinner atomizers to be inserted therein.

Advantageously, by keeping of the atomizer away from contacting the users skin or clothing by having it in the housing and at least partially enclosed therein allows for proving of protection to the user from being burnt by a potentially hot atomizer. Optionally the at least one electrical switch 108a also advantageously allows for the device not to trigger by accident when in the pocket.

Numerous other embodiments may be envisaged without departing from the spirit or scope of the invention.

What I claim is:

1. A vaporization device for vaporizing of phyto material comprising:
an atomizer comprising a heating chamber for receiving of the phyto material, the atomizer comprising a inhalation aperture disposed at a first end thereof and a coupling mechanism disposed at a second end thereof, opposite the first end,
the atomizer for operating in a first mode of operation—a closed mode—and a second mode of operation—a second mode,
the atomizer comprising a heating element disposed within the heating chamber, the heating element for providing of heat to the phyto material using at least one of convection and conduction;
a housing having a first portion and a second portion, the first portion having a recessed channel that is open on at least one side for receiving of the atomizer substantially disposed therein in the first mode of operation and for having the atomizer other than disposed within the first portion in the second mode of operation;
a rotating member having a rotational axis and rotationally coupled with the housing for rotating about the rotational axis between the first and second modes of operation;
a pushbutton switch mechanically coupled with the housing and the rotating member, the pushbutton switch for facilitating movement of the rotating member about the rotational axis and for locking of the rotating member in its position in at least one of the first and second modes of operation, wherein the first mode of operation the rotating member is approximately at a zero degrees orientation with respect to the housing and in the second mode of operation the rotating member is at a greater than forty five degrees orientation with respect to the housing;
a battery and a control circuit disposed within the housing the control circuit electrically coupled with the battery and the coupling mechanism.

2. A vaporization device for vaporizing of phyto material according to claim 1 comprising: at least one electrical switch disposed between the housing and the rotating member, the at least one electrical switch electrically disposed between the control circuit and the coupling mechanism, the electrical switch for being in an electrically opened position when in the first mode of operation where electric current other than flows from the control circuit to the heating element and in an electrically closed position in the second mode of operation where electric current flows from the control circuit to the heating element.

3. A vaporization device for vaporizing of phyto material according to claim 1 wherein the coupling mechanism comprises: a first electrical contact a second electrical contact, wherein the first electrical contact comprises a ground connection and the second electrical contact comprises a positive connection from the control circuit.

4. A vaporization device for vaporizing of phyto material according to claim 3 wherein the coupling mechanism comprises a third electrical contact and comprises the atomizer comprising a temperature sensor disposed proximate the heating element for sensing a temperature of the heating element and for providing of a temperature signal through the third electrical contact to the control circuit for varying a power provided from the battery to the heating element.

5. A vaporization device for vaporizing of phyto material according to claim 1 wherein the coupling mechanism comprises a thread for threaded coupling of the atomizer to the rotating member.

6. A vaporization device for vaporizing of phyto material according to claim 1 wherein the coupling mechanism comprises other than a thread and comprises a magnetic coupling for magnetic coupling of the atomizer to the rotating member.

7. A vaporization device for vaporizing of phyto material according to claim 1 comprising a first air gap formed between atomizer and the housing first portion in the first mode of operation, wherein the at least a first air gap shields the user in conjunction with the housing first portion from a hot atomizer after use when the vaporization device is placed in a pocket of the user.

8. A vaporization device for vaporizing of phyto material according to claim 7 comprising a second air gap formed between atomizer and the housing first portion in the first mode of operation, wherein the second air gap shields the user in conjunction with the housing first portion from an opposite side of the first air gap from a hot atomizer after use when the vaporization device is placed in a pocket of the user.

9. A vaporization device for vaporizing of phyto material according to claim 1 comprising a phyto material storage container disposed as part of the housing second portion for storing of phyto material therein and having a removable lid for accessing of the phyto material stored therein for being loaded into the heating chamber.

10. A vaporization device for vaporizing of phyto material according to claim 1 comprising a pick tool for being disposed within the housing second portion, the pick tool for at least one of stirring the phyto material within the heating chamber and for removing of spent phyto material from the heating chamber.

11. A vaporization device for vaporizing of phyto material according to claim 1 comprising a spring, the spring for being compressed in the first mode of operation when the atomizer travels from the second mode of operation back to the first mode of operation and the spring pressing against a pushbutton switch against the rotating member upon the pushbutton switch being activated by the user it moves away from a locking portion of the rotating member and spring releases stored energy and the rotating member rotates about its rotational axis and the atomizer leaves the housing first portion as the second mode of operation.

12. A vaporization device for vaporizing of phyto material comprising:
an atomizer comprising a heating chamber for receiving of the phyto material, the atomizer comprising a inhalation aperture disposed at a first end thereof and a coupling mechanism disposed at a second end thereof, opposite the first end,
the atomizer for operating in a first mode of operation—a closed mode—and a second mode of operation—a second mode,
the atomizer comprising a heating element disposed within the heating chamber, the heating element for providing of heat to the phyto material using at least one of convection and conduction;
a housing having a first portion and a second portion, the first portion having a recessed channel that is open on at least one side for receiving of the atomizer substantially disposed therein in the first mode of operation and for having the atomizer other than disposed within the first portion in the second mode of operation;
a rotating member having a rotational axis and rotationally coupled with the housing for rotating about the rotational axis between the first and second modes of operation;
a pushbutton switch mechanically coupled with the housing and the rotating member, the pushbutton switch for facilitating movement of the rotating member about the rotational axis and for locking of the rotating member in its position in at least one of the first and second modes of operation, wherein the first mode of operation the rotating member is approximately at a zero degrees orientation with respect to the housing and in the second mode of operation the rotating member is at a greater than forty five degrees orientation with respect to the housing;
a battery and a control circuit disposed within the housing the control circuit electrically coupled with the battery and the coupling mechanism;
at least one electrical switch disposed between the housing and the rotating member, the at least one electrical switch electrically disposed between the control circuit and the coupling mechanism, the electrical switch for being in an electrically opened position when in the first mode of operation where electric current other than flows from the control circuit to the heating element and in an electrically closed position in the second mode of operation where electric current flows from the control circuit to the heating element.

13. A vaporization device for vaporizing of phyto material comprising:
an atomizer comprising a heating chamber for receiving of the phyto material, the atomizer comprising a inhalation aperture disposed at a first end thereof and a coupling mechanism disposed at a second end thereof, opposite the first end,
the atomizer for operating in a first mode of operation—a closed mode—and a second mode of operation—a second mode,
the atomizer comprising a heating element disposed within the heating chamber, the heating element for providing of heat to the phyto material using at least one of convection and conduction;
a housing having a first portion and a second portion, the first portion having a recessed channel that is open on at least one side for receiving of the atomizer substantially disposed therein in the first mode of operation and for having the atomizer other than disposed within the first portion in the second mode of operation;
a rotating member having a rotational axis and rotationally coupled with the housing for rotating about the rotational axis between the first and second modes of operation;
a pushbutton switch mechanically coupled with the housing and the rotating member, the pushbutton switch for facilitating movement of the rotating member about the rotational axis and for locking of the rotating member in its position in at least one of the first and second modes of operation, wherein the first mode of operation the rotating member is approximately at a zero degrees orientation with respect to the housing and in the second mode of operation the rotating member is at a greater than forty five degrees orientation with respect to the housing;
a battery and a control circuit disposed within the housing the control circuit electrically coupled with the battery and the coupling mechanism;
a first electrical contact a second electrical contact, wherein the first electrical contact comprises a ground connection and the second electrical contact comprises a positive connection from the control circuit; and,
a third electrical contact and wherein the atomizer comprises a temperature sensor disposed proximate the heating element for sensing a temperature of the heating element and for providing of a temperature signal through the third electrical contact to the control circuit for varying a power provided from the battery to the heating element.
